(19) United States
(12) Patent Application Publication
(10) Pub. No.: US 2017/0003299 A1
(43) Pub. Date: Jan. 5, 2017

(54) DETECTING PODOCYTE INJURY IN DIABETIC NEPHROPATHY AND GLOMERULONEPHRITIS

(71) Applicant: Mayo Foundation for Medical Education and Research, Rochester, MN (US)

(72) Inventors: Vesna D. Garovic, Rochester, MN (US); Muthuvel Jayachandran, Rochester, MN (US)

(73) Assignee: Mayo Foundation for Medical Education and Research, Rochester, MN (US)

(21) Appl. No.: 15/100,136

(22) PCT Filed: Nov. 5, 2014
(86) PCT No.: PCT/US14/64007

§ 371 Date: May 27, 2016

Related U.S. Application Data
(60) Provisional application No. 61/909,888, filed on Nov. 27, 2013.

(51) Int. Cl. G01N 33/68

(52) U.S. Cl. CPC G01N 33/6893; G01N 2333/4742; G01N 2333/47; G01N 2333/71; G01N 2333/70596; G01N 2333/70564; G01N 2800/347

(57) ABSTRACT

This document provides methods and materials involved in determining whether or not a mammal with diabetic nephropathy or glomerulonephritis is undergoing podocyte injury. For example, methods and materials related to the use of urinary microvesicles to determine whether or not a mammal with diabetic nephropathy or glomerulonephritis is undergoing podocyte injury are provided.

### CROSS-REFERENCE TO RELATED APPLICATIONS

This application claims the benefit of U.S. Provisional Application Ser. No. 61/909,888, filed Nov. 27, 2013. The disclosure of the prior application is considered part of (and is incorporated by reference in) the disclosure of this application.

### BACKGROUND

1. Technical Field

This document relates to methods and materials involved in determining whether or not a mammal with diabetic nephropathy or glomerulonephritis is undergoing podocyte injury. For example, this document provides methods and materials related to the use of urinary microvesicles to determine whether or not a mammal with diabetic nephropathy or glomerulonephritis is undergoing podocyte injury.

2. Background Information

Microvesicles are small (e.g., about 0.1 to 1 μm) membrane-enclosed sacs that are shed from activated or injured cells and contribute to a variety of pathophysiological processes. They are involved in intercellular communication either locally or at a distance primarily by transferring the cytosolic content of one cell to another.

### SUMMARY

This document provides methods and materials for determining whether or not a mammal with diabetic nephropathy or glomerulonephritis is undergoing podocyte injury. For example, this document provides methods and materials related to the use of urinary microvesicles to determine whether or not a mammal with diabetic nephropathy or glomerulonephritis is undergoing podocyte injury. In some cases, the detection of urinary microvesicles as described herein can be used to diagnose a mammal as having diabetic nephropathy, glomerulonephritis, diabetic nephropathy with podocyte injury, or glomerulonephritis with podocyte injury.

Identifying patients who have diabetes or glomerulonephritis with podocyte injury can allow such patients, who are at risk of progressive renal injury, to be treated effectively. In addition, identifying patients who do not have podocyte injury can avoid unnecessary treatment and patient suffering. As described herein, the presence of urinary microvesicles can be used to identify mammals (e.g., humans) as having diabetic nephropathy, glomerulonephritis, diabetic nephropathy with podocyte injury, or glomerulonephritis with podocyte injury.

In general, one aspect of this document features a flow cytometry method or enzyme-linked immunosorbent assay method for detecting diabetic nephropathy or glomerulonephritis. The method comprises, or consists essentially of, (a) performing flow cytometry or an enzyme-linked immunosorbent assay using a urine sample obtained from a mammal to detect the presence of an elevated level of urinary microvesicles expressing a podocyte, parietal, mesangial, or endothelial cell marker, and (b) classifying the mammal as having diabetic nephropathy or glomerulonephritis. The mammal can be a human. The method can comprise detecting the presence of an elevated level of urinary microvesicles expressing a podocyte cell marker. The podocyte cell marker can be synaptopodin, nephrin, podocin, or podocalyxin. The method can comprise detecting the presence of an elevated level of urinary microvesicles expressing a parietal cell marker. The parietal cell marker can be claudin-1 or CK-8. The method can comprise detecting the presence of an elevated level of urinary microvesicles expressing a mesangial cell marker. The mesangial cell marker can be PDGFRβ or α-SMA. The method can comprise detecting the presence of an elevated level of urinary microvesicles expressing an endothelial cell marker. The endothelial cell marker can be CD62E or CD31.

In another aspect, this document features a method for detecting podocyte injury in a mammal having a diabetic nephropathy or glomerulonephritis. The method comprises, or consists essentially of, (a) detecting, in a urine sample obtained from the mammal, the presence of an elevated level of urinary microvesicles expressing a podocyte, parietal, mesangial, or endothelial cell marker, and (b) classifying the mammal as having podocyte injury. The mammal can be a human. The method can comprise detecting the presence of an elevated level of urinary microvesicles expressing a podocyte cell marker. The podocyte cell marker can be synaptopodin, nephrin, podocin, or podocalyxin. The method can comprise detecting the presence of an elevated level of urinary microvesicles expressing a parietal cell marker. The parietal cell marker can be claudin-1 or CK-8. The method can comprise detecting the presence of an elevated level of urinary microvesicles expressing a mesangial cell marker. The mesangial cell marker can be PDGFRβ or α-SMA. The method can comprise detecting the presence of an elevated level of urinary microvesicles expressing an endothelial cell marker. The endothelial cell marker can be CD62E or CD31. The method can comprise performing a flow cytometry assay or an enzyme-linked immunosorbent assay to detect the presence.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-E are representative scatter plots obtained by FACSCanto™ flow cytometry. FIG. 1A: Control gates of buffer with fluorescein conjugated antibodies and calibration (size and TruCount™ Beads) beads in the absence of sample. FIG. 1B: Gates derived from adding a sample containing microvesicles (MV) to the buffer with fluorescein conjugated antibodies and calibration TruCount™ beads. Note that the gate to define the area of interest (R1) was set above the background noise of the instrument. FIGS. 1C-E involved a urine sample from a patient with diabetes mellitus. FIG. 1C: Urinary MV in the absence of any antibodies (sample only). FIG. 1D: Urinary MV in the presence of FITC-conjugated synaptopodin (Q4 showing synaptopodin positive MV). FIG. 1E: Urinary MV in the presence of PE-conjugated podocin (Q1 showing podocin positive MV).

FIG. 2 is a larger version of FIG. 1B.

FIGS. 3-6 contains scatter plots obtained by flow cytometry using the indicated markers.

FIG. 7 is a graph plotting the association between urinary albumin to creatinine (mg/g) to the number of urinary MVs positive for annexin-V in the GN group (r²=0.42, P=0.056).

### DETAILED DESCRIPTION

This document provides methods and materials related to determining whether or not a mammal (e.g., a human) with diabetic nephropathy or glomerulonephritis is undergoing podocyte injury. For example, this document provides methods and materials related to the use of urinary microvesicles to determine whether or not a mammal (e.g., a human) with diabetic nephropathy or glomerulonephritis is undergoing podocyte injury. As described herein, if the level of urinary microvesicles is elevated in a mammal with diabetic nephropathy or glomerulonephritis, then the mammal can be classified as having podocyte injury. If the level of urinary microvesicles is not elevated in a mammal with diabetic nephropathy or glomerulonephritis, then the mammal can be classified as not having podocyte injury.

Any appropriate urinary microvesicle can be used as described herein to determine whether or not a mammal (e.g., a human) with diabetic nephropathy or glomerulonephritis is undergoing podocyte injury. For example, urinary microvesicles expressing a podocyte, parietal, mesangial, or endothelial cell marker can be used. Examples of urinary microvesicles expressing a podocyte cell marker include, without limitation, those urinary microvesicles expressing synaptopodin, nephrin, podocin, or podocalyxin. Examples of urinary microvesicles expressing a parietal cell marker include, without limitation, those urinary microvesicles expressing claudin-1 or CK-8. Examples of urinary microvesicles expressing a mesangial cell marker include, without limitation, those urinary microvesicles expressing PDGFRβ or α-SMA. Examples of urinary microvesicles expressing an endothelial cell marker include, without limitation, those urinary microvesicles expressing CD62E or CD31.

Microvesicles, which can be present in urine, are a heterogeneous population of spheres (varying in size from about 0.1 to 1.0 μm) formed from intact phospholipid rich membranes. Typically, a microvesicle contains at least half of the surface polypeptides, receptors, and lipids of their cells of origin. Microvesicles can be generated during cell activation and apoptosis induced by oxidative damage, inflammatory cytokines and chemokines, thrombin, bacterial lipopolysaccharide, shear stress, and hypoxia.

Once obtained, a urine sample can be analyzed using antibodies in an ELISA or flow cytometry assay (e.g., flow cytometry assay based on size) to determine the total number of microvesicles, the level of microvesicles of a particular cellular origin, or the level urinary microvesicles expressing a podocyte, parietal, mesangial, or endothelial cell marker, present within a urine sample. For example, a FACSCanto™ (New fourth or fifth generation) machine with high sensitivity and six colors detectors can be used to detect urinary microvesicles. In some cases, digital flow cytometry can be used to detect urinary microvesicles.

The term “elevated level” as used herein with respect to the level of urinary microvesicles (e.g., urinary microvesicles expressing a podocyte cell marker) is any level that is above a median urinary microvesicle level in urine from a random population of healthy mammals (e.g., a random population of 10, 20, 30, 40, 50, 100, or 500 healthy humans) lacking podocyte injury. In some cases, an elevated level of urinary microvesicles (e.g., urinary microvesicles expressing a podocyte cell marker) can be any level that is greater than a reference level for urinary microvesicles (e.g., urinary microvesicles expressing a podocyte cell marker). The term “reference level” as used herein with respect to a level of urinary microvesicles (e.g., urinary microvesicles expressing a podocyte cell marker) is the level of urinary microvesicles (e.g., urinary microvesicles expressing a podocyte cell marker) typically found in healthy mammals, for example, mammals free of signs and symptoms of podocyte injury. For example, a reference level of urinary microvesicles (e.g., urinary microvesicles expressing a podocyte cell marker) can be the average level of urinary microvesicles (e.g., urinary microvesicles expressing a podocyte cell marker) that is present in urine samples obtained from a random sampling of 50 healthy mammals matched for age. It will be appreciated that levels from comparable samples are used when determining whether or not a particular level is an elevated level.

An elevated level of urinary microvesicles (e.g., urinary microvesicles expressing a podocyte cell marker) can be any level provided that the level is greater than a corresponding reference level for urinary microvesicles. For example, an elevated level of urinary microvesicles can be 2, 3, 4, 5, 6, 7, 8, 9, 10, 15, 20, or more times greater than the reference level for urinary microvesicles.

In some cases, antibodies can be used as described herein to determine the source of urinary microvesicles. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-podocin antibody can bind to podocin polypeptides at an affinity of at least 10⁴ mol⁻¹ (e.g., at least 10⁵, 10⁶, 10⁷, 10⁸, 10⁹, 10¹⁰, 10¹¹, or 10¹² mold).

The invention will be further described in the following examples, which do not limit the scope of the invention described in the claims.

### EXAMPLES

### Example 1

### Using Urinary Microvesicles to Detect Podocyte Injury in Diabetic Nephropathy and Glomerulonephritis

### Patients and Urine Samples

Urine samples from patients diagnosed with type 2 diabetes mellitus (DM) (n=31), glomerulonephritis (GN) (n=9), and healthy controls (n=10) were used for identification of urinary microvesicles. Patients were diagnosed with type 2 DM if they were receiving an oral hypoglycemic agent or insulin. All other etiologies that may result in proteinuric renal disease including autoimmune diseases, hepatitis B or C, antineutrophil cytoplasmic antibody (ANCA), anti-glomerular basement membrane (GBM) vasculitides, or monoclonal gammopathy were excluded. GN diagnoses were based on renal biopsy findings. Five patients had a diagnosis of membranous nephropathy (MN), and four patients had primary focal segmental glomerulosclerosis (FSGS). Expect for one patient with MN who was in partial remission, all patients had active disease. Healthy controls were those who were approved for kidney donation, and urine samples were obtained prior to the kidney donation. Patients with estimated glomerular filtration rate (eGFR) less than 15 mL/min/1.73 m² were excluded.

All patients had a serum creatinine measurement (a measure of renal function) within one week of urine collection, and all diabetic patients had a serum HgbA1C measurement (a marker of disease control) within four weeks of urine collection. All patients underwent a measurement of blood pressure and heart rate within two days of urine collection, and each patient's weight and height was determined within four weeks of urine collection.

Urine samples were random spot urines and were processed within one hour of collection. Fifty mL of each urine sample were centrifuged at room temperature at 1750 rpm for 10 minutes. The supernatant was then frozen immediately at −80° C. A portion of the urine supernatant was used to measure urinary albumin, protein, and creatinine levels.

### Antibodies and Other Reagents

Annexin-V and mouse anti-human CD62E, CD31, and CD63 with fluorescein isothiocyanate (FITC) or phycoerythrin (PE) and TruCOUNT™ (4.2 μm) beads were purchased from BD Biosciences (San Jose, Calif.). Rabbit anti-human synaptopodin, nephrin, podocin, claudin-1, and polyclonal immunoglobulin (Ig) G isotype labeled with FITC or PE were purchased from Bioss (Woburn, Mass.). Mouse anti-human podocalyxin, platelet derived growth factor-β (PDGFRβ) and monoclonal IgG2A labeled with fluoro 488 or PE were purchased from R&D Systems (Minneapolis, Minn.). Mouse anti-human cytokeratin (CK)-8 and α-smooth muscle actin (SMA) labeled with FITC were purchased from Abcam (Cambridge, Mass.). Monoclonal IgG1 isotype was purchased from Miltenyi Biotec (Auburn, Calif.).

### Flow Cytometry

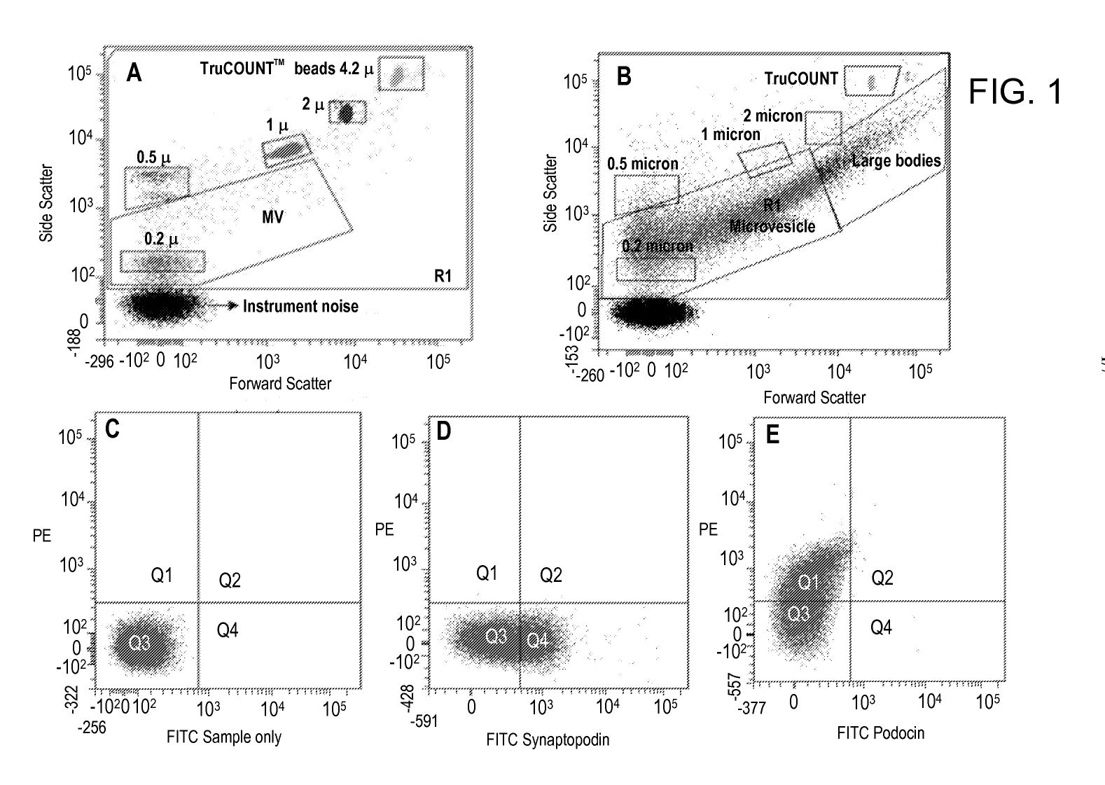

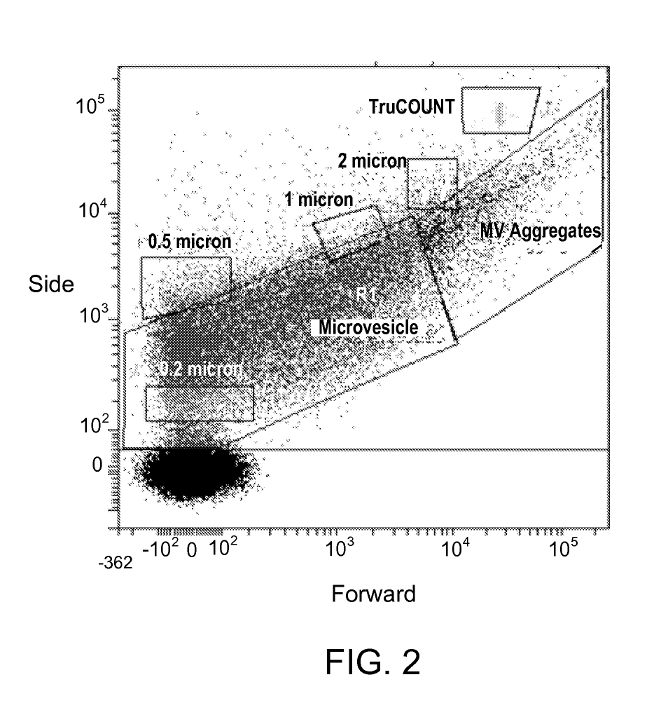

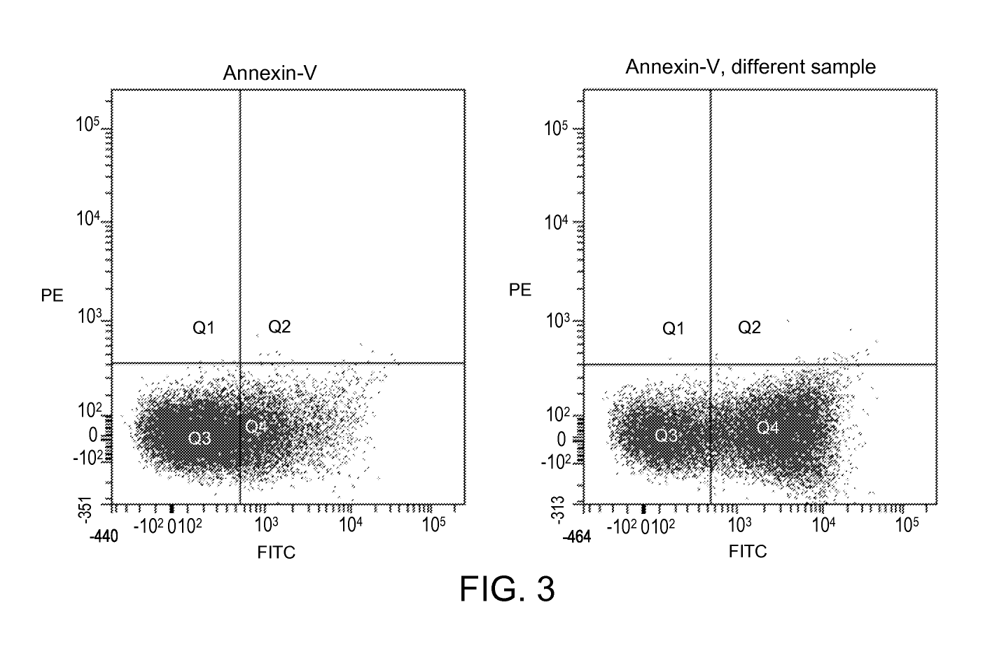

All analyses used a FACSCanto™ cytometer (BD Biosciences, San Jose, Calif.). All antibodies and the Hank's/HEPES (H/H) buffer (pH7.4) were filtered twice through 0.2 μm membrane filters as unfiltered buffers and antibodies have interfering chemical microparticles. Microvesicles were defined as events <1 μm in diameter and positive for annexin-V or specific cell-markers. Urine sample (20 μL) after addition of 80 μL of H/H buffer was incubated with 5 μL of annexin-V-FITC for 30 minutes Immediately prior to running the sample, additional 900 μL of H/H buffer was added to the sample. The amount of urine used in the subsequent experiments was adjusted in order to have at least 1500 microvesicles that stained positive with Annexin-V and at least 10,000 total events (FIGS. 1-3). Annexin-V binds to phosphatidyl serine that is expressed on the surface of microvesicles and allows their detection amongst microvesicles in the urine (Zwaal et al., Blood, 89:1121-1132 (1997); Jayachandran et al., J. Immunol. Methods, 375:207-214 (2012); and Koopman et al., Blood, 84:1415-1420 (1994)).

After identifying the amount of urine required for the experiment (ranging between 20-80 μL) based on the annexin-V positive events, the urine was incubated for 30 minutes with different cellular markers. Monoclonal antibodies were not diluted, but polyclonal antibodies were diluted 1:10 to decrease nonspecific binding. Synaptopodin, nephrin, podocin, and podocalyxin were used as podocyte markers. Claudin-1 and CK-8 were used as parietal cell markers (Kiuchi-Saishin et al., J. Am. Soc. Nephrol., 13:875-886 (2002); Ohse et al., J. Am. Soc. Nephrol., 19:1879-1890 (2008); and Dijkman et al., Kidney Int., 68:1562-1572 (2005)). PDGFRβ and α-SMA were used as mesangial cell markers (Sarrab et al., Am. J Physiol. Renal Physiol., 301:F1131-1138 (2011) and Takano et al., Tohoku J. Exp. Med., 212:81-90 (2007)). CD31 and CD62e were used as markers of endothelial cells. For all experiments, 5 μL of the antibody was used. The thresholds were set with isotype control antibodies. For calculation of counts, TruCOUNT™ beads (100 μL) were added immediately prior to analysis by flow cytometry. The absolute number of microvesicles was calculated as described elsewhere (Jayachandran et al., J. Immunol. Methods, 375:207-214 (2012)). The specificity of the podocyte cell markers were confirmed by the presence of microvesicles positive for synaptopodin, podocin, nephrin, and podocalyxin when using the media in which a human podocyte cell line (Saleem et al., J. Am. Soc. Nephrol., 13:630-638 (2002)) were cultured.

### Statistical Analysis

Unless otherwise indicated, the data were summarized as median with range from minimum to maximum. Numerical data were compared using nonparametric test (Kruskall-Wallis). P<0.05 was deemed significant.

### Results

### Baseline Patient Characteristics

Total of 50 patients were recruited. Thirty one patients had type 2 DM and were further divided into three groups: Group 1 with urine albumin to creatinine ratio <30 mg/g (n=13), Group 2 with urine albumin to creatinine ratio between 30-300 mg/g (n=10), and Group 3 with albumin to creatinine ratio >300 mg/g (n=8). Nine patients had GN of which five had MN and four had FSGS. Ten patients were healthy controls. Patients' baseline demographics are summarized in Table 1.

TABLE 1
Baseline patient characteristics.

| | Healthy | | | | | |
|---|---|---|---|---|---|---|
| | Control | | | | | |
| Parameters | Group | Group 1 | Group 2 | Group 3 | GN Group | P value |
| n | 10 | 15 | 7 | 9 | 9 | |
| Alb/Cr | 6 | 10 | 55 | 1064 | 3028 | <0.0001^{a} |
| (mg/g) | (1-15) | (2-29) | (32-269) | (318-3366) | (833-6999) | |
| Age (years) | 42 | 61 | 62 | 64 | 52 | 0.0023^{b} |
| | (30-56) | (26-78) | (51-81) | (52-81) | (23-66) | |
| Gender | 5/5 | 8/7 | 4/3 | 7/2 | 6/3 | 0.7 |
| (M/F) | | | | | | |
| SBP (mmHg) | 113 | 129 | 132 | 128 | 137 | 0.02^{c} |
| | (101-128) | (101-139) | (116-159) | (109-161) | (108-147) | |
| DBP | 70 | 75 | 64 | 59 | 68 | 0.1 |
| (mmHg) | (62-79) | (68-88) | (57-81) | (55-93) | (65-90) | |
| BMI (Kg/m²) | 28 | 31 | 31 | 33 | 30 | 0.2 |
| | (21-32) | (23-41) | (21-51) | (29-41) | (27-38) | |
| Serum | 0.7 | 0.6 | 1.2 | 1.0 | 1.6 | 0.003^{d} |
| creatinine | (0.6-1.1) | (0.5-1.5) | (0.9-1.6) | (0.7-3.0) | (0.9-2.6) | |
| (mg/dL) | | | | | | |
| eGFR | 88 | 82 | 56 | 75 | 43 | 0.004^{e} |
| (mL/min/1.73 m²) | (65-109) | (45-137) | (34-89) | (18-89) | (27-69) | |
| Use of ACE- | 0/10 | 7/8 | 6/1 | 7/2 | 7/2 | 0.0001 |
| I or ARB | | | | | | |
| (Yes/No) | | | | | | |
| Diabetic | 0/0 | 10/5 | 6/1 | 4/5 | 0/0 | 0.2 |
| medication | | | | | | |
| (oral/insulin) | | | | | | |

^{a}= all groups were significantly different except for healthy controls compared to Group 1.
^{b}= all groups were significantly different compared to healthy controls. P = 0.05 (GN. vs. Group 3)
^{c}= Healthy controls vs. Group 2 and 3 were significantly different
^{d}= Healthy controls significantly different compared to Group 2, 3, and GN.
^{e}= GN group different compared to Healthy control and Group 1.

There was a significant difference in the urinary albumin to creatinine ratio amongst the five groups (P<0.0001), but importantly there was no difference between the healthy controls and Group 1. Urinary albumin to creatinine ratio was significantly higher in the GN group compared to Group 3 (P=0.03). Healthy controls were significantly younger compared to all other four groups (p=0.002) and had lower serum creatinine (P=0.003) and higher estimated glomerular filtration rate (eGFR) (P=0.004) and lower systolic blood pressure (BP) (P=0.02) compared to Group 2, Group 3, and the GN group. There was no difference in systolic BP, serum creatinine, and eGFR between healthy controls and Group 1 (Table 1).

### Podocyte-Specific Markers

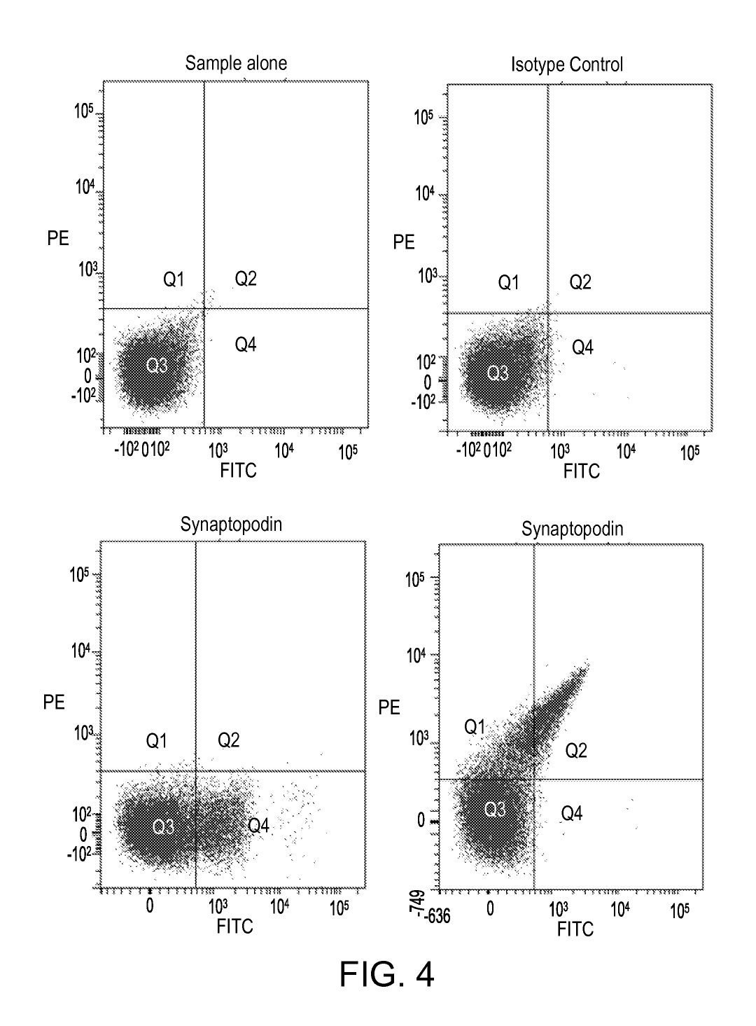

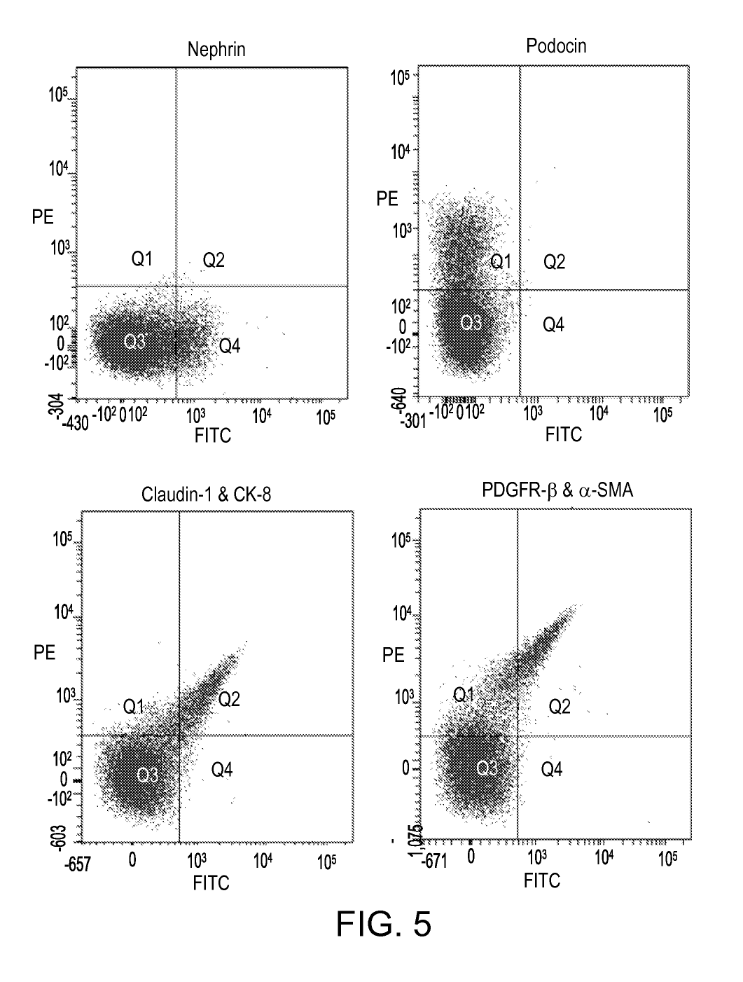

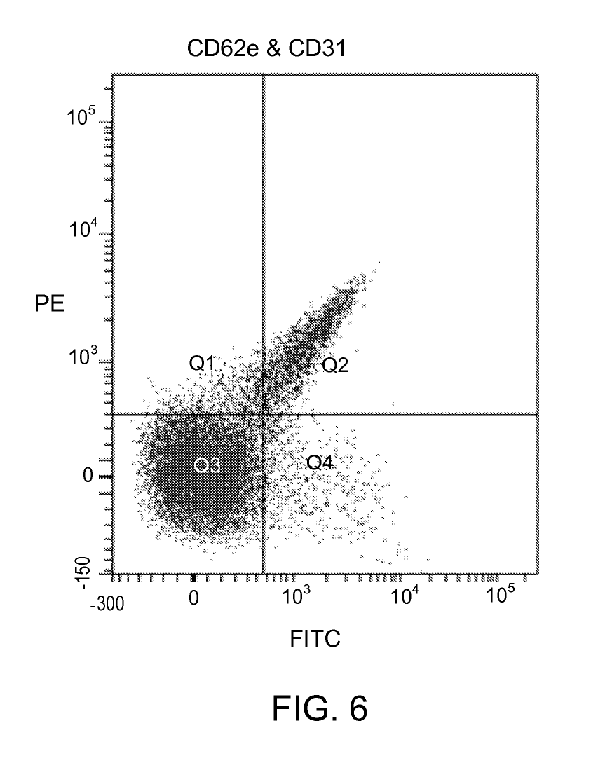

The urinary microvesicles were evaluated for expression of podocyte-specific markers (synaptopodin, podocin, nephrin, and podocalyxin), parietal cell markers (claudin-1 and CK-8), mesangial cell markers (PDGFR-β and α-SMA), and endothelial cell markers (CD62E and CD31) representing cells of the entire glomerulus. FIGS. 4-6 are examples of flow cytometry results for each marker used.

Overall there were significantly higher expression of podocyte-specific markers including nephrin (p=0.02), podocalyxin (P=0.0001), synaptopodin (P<0.0001), and podocin (P<0.0001) in all diabetic patients compared to the healthy controls. Specifically, the number of urinary microvesicles in Group 1 (non-albuminuric type 2 DM) expressing nephrin (568/μL (39-2424)), podocalyxin (838/μL (111-1181), synaptopodin (1074/μL (306-3210)), and podocin 1454/μL (266-3230) were significantly higher than healthy controls (Table 2).

TABLE 2
Number of microvesicles expressing specific markers in each group.

| | Healthy | | | | | |
|---|---|---|---|---|---|---|
| | Control | | | | | |
| | Group | Group 1 | Group 2 | Group 3 | GN Group | P value |
| n | 10 | 15 | 7 | 9 | 9 | |
| Nephrin | 22 | 568 | 241 | 317 | 618 | 0.02^{a} |
| | (2-205) | (39-2424) | (111-1181) | (89-663) | (174-1050) | |
| Podocalyxin | 10 | 838 | 777 | 558 | 1377 | 0.0001^{a} |
| | (3-280) | (231-3443) | (372-3480) | (163-1747) | (543-2922) | 0.02^{b} |
| Synaptopodin | 33 | 1074 | 610 | 843 | 1250 | <0.0001^{a} |
| | (4-270) | (306-3210) | (276-3125) | (215-1627) | (870-2430) | 0.06^{b} |
| Podocin | 37 | 1454 | 911 | 1261 | 1268 | 0.001^{a} |
| | (9-1177) | (266-3230) | (224-3623) | (254-4839) | (591-3966) | 0.9^{b} |
| Claudin-1/ | 3 | 479 | 340 | 299 | 761 | <0.0001^{a} |
| Ck-8 | (2-296) | (48-1813) | (87-1191) | (75-670) | (302-1607) | 0.002^{b} |
| PDGFR-β/ | 6 | 555 | 608 | 271 | 793 | <0.0001^{a} |
| α-SMA | (0-182) | (85-2614) | (138-2420) | (90-1642 | (258-2116) | 0.01^{b} |
| CD62E/ | 1 | 550 | 504 | 281 | 744 | <0.0001^{a} |
| CD31 | (0-138) | (54-2463) | (114-1793) | (42-1524) | (257-2652) | 0.01^{b} |

^{a}= Healthy control group compared to Group 1, 2, 3, and GN group
^{b}= GN group vs. Group 3

The number of microvesicles expressing nephrin in GN group was 618/μL of urine (174-1050), which was significantly higher than 317/μL (89-663) in Group 3 (P=0.02). Similarly, there were significantly higher number of microvesicles expressing podocalyxin in GN group compared to Group 3 (type 2 DM with macroalbuminuria) (P=0.027, Table 2). A similar trend was present when comparing microvesicles expressing synaptopodin (P=0.06) in the GN group to Group 3, but no significant difference was noted when evaluating podocin (P=0.9).

### Parietal, Mesangial, and Endothelial Cell Markers

Microvesicles were considered to be from parietal cell origin if they were expressing both claudin-1 and CK-8 (Kiuchi-Saishin et al., J. Am. Soc. Nephrol., 13:875-886 (2002); Ohse et al., J. Am. Soc. Nephrol., 19:1879-1890 (2008); and Dijkman et al., Kidney Int., 68:1562-1572 (2005)). Similar to the podocyte-specific markers, there were minimal urinary microvesicles expressing parietal cell markers (33/μL (2-296), which was significantly lower than all other groups including Group 1 (479/pt (48-1813)). GN group had the highest expression of parietal cell markers (761/μL (302-1607), and even though not significantly different than Group 1 and 2, it was significantly higher than Group 3 (299/μL (75-670)) (P=0.002).

Microvesicles expressing mesangial cell markers including both PDGFR-β and α-SMA (Sarrab et al., Am. J. Physiol. Renal Physiol., 301:F1131-1138 (2011) and Takano et al., Tohoku J. Exp. Med., 212:81-90 (2007)) were significantly lower in the healthy controls (6/μL (0-182)) compared to other four groups (P<0.0001). In addition, mesangial markers were higher in the GN group (793/μL (258-2116)) compared to Group 3 (271/μL (90-1642)) (P=0.01). Similarly, microvesicles expressing markers for endothelial cells including both CD62e (marker of activated endothelial cells; Jayachandran et al., J. Immonol. Methods, 375:207-214 (2012)) and CD31 were significantly higher in all four groups compared to healthy controls (P<0.0001) and similarly higher in GN group (744/μL (257-2652) compared to Group 3 (281/μL (42-1524)) (P=0.01).

### Urinary Microvesicles and Clinical/Laboratory Data

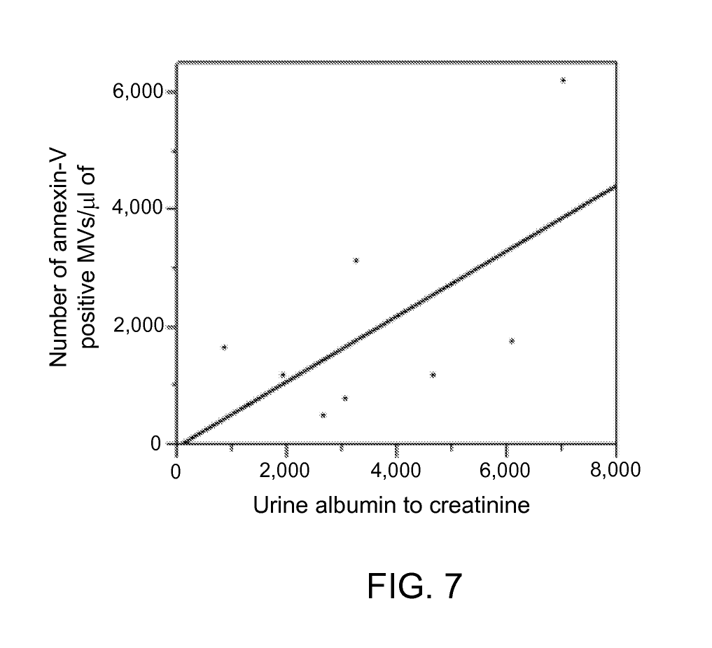

The potential association between the number of urinary microvesicles (positive for annexin-V) and other clinical/laboratory data was evaluated. There was no association between total number of annexin-V positive urinary microvesicles with age, gender, blood pressure (systolic or diastolic), BMI, and HgbA1C in each group or overall. When all groups were combined, there was no association between urinary albumin to creatinine ratio and total number of urinary microvesicles. There was a trend towards significance when comparing urinary albumin level and annexin-v positive microvesicles in GN group (P=0.05) (FIG. 7). In addition, neither of the glomerular markers exhibited any significant correlation with the above-mentioned factors. Overall there was no difference between the stage of chronic kidney disease (CKD) and the number of urinary microvesicles. The four patients with stage 4 CKD exhibited overall lower numbers of microvesicles, two of which were diabetic patients with macroalbuminuria and two of which were patients with GN, but this did not reach statistical significance.

These results demonstrate that digital flow cytometry can be used to evaluate podocyte injury by detecting microvesicles expressing glomerular markers in the urine of patients with type 2 DM and GN compared to the healthy controls. For example, a higher number of urinary microvesicles expressing podocyte-specific, parietal, mesangial, and endothelial cell markers were detected in patient with type 2 DM and GN compared to the healthy controls. Patients in the GN group exhibited the highest number of urinary microvesicles for all glomerular markers compared to the other groups. Particularly, this difference was significant when comparing the GN group to Group 3, which had the highest urinary albumin level compared to the other diabetic patients (Groups 1 and 2). The difference between the GN group and Group 3 was most prominent when comparing the number of nephrin positive urinary microvesicles. This difference was no longer present when evaluating podocin positive microvesicles. These findings may reflect the fact that the expression of nephrin decreases as diabetic nephropathy becomes more advanced.

The absence of a difference in podocin expression between Group 3 and GN group may reflect continued expression of podocin in advanced diabetic nephropathy. This differential expression of nephrin may be used as a diagnostic marker to help identify those diabetic patients who may have an underlying GN rather than or in addition to their diabetic nephropathy, as not all patients with diabetic nephropathy have evidence of diabetic involvement on renal biopsy and nondiabetic renal diseases such as MN or FSGS may account for the proteinuria in this population.

The higher expression of podocyte-specific markers was also noted in Group 1-3 compared to the healthy controls. Most importantly, Group 1 (DM without albuminuria) had higher expression of nephrin, podocin, synaptopodin, and podocalyxin compared to the healthy controls. These findings suggest that podocyte injury occurs prior to development of proteinuria. It should be noted that not all patients in Group 1 had high expression of these markers. If 500 microvesicles/μL of urine is used as a cutoff above which the expression would be considered abnormal, then 46% of patients in Group 1 exhibited abnormal expression of nephrin positive microvesicles. It was hypothesized that patients with high expression of podocyte-specific markers may be at higher risk of developing diabetic nephropathy long-term. Age, gender, serum creatinine, eGFR, systolic BP, BMI, and HgbA1c did not have any association with the number of urinary microvesicles in each group or overall indicating that the driving factor for shedding of the urinary microvesicles from podocyte is the underlying disease.

A higher number of urinary microvesicles expressing parietal cell markers also was identified in the GN and diabetic groups (particularly Group 1) compared to the healthy controls. The expression of parietal cell markers was lowest in Group 3 and highest in Group 1 and the GN group. This likely reflects the active process of proliferation of parietal cells and attempts at regenerating podocytes in the GN group as opposed to the advanced diabetic group in which this process may be less active. The high number of microvesicles expressing parietal cells markers in non-albuminuric DM patients (Group 1) was intriguing. Despite the absence of proteinuria, this group exhibited the highest expression of CK-8 and Claudin-1 compared to the healthy controls. This suggests that injury to the podocytes occurs prior to development of proteinuria, and the active process of podocyte regeneration may be taking place early on in the course of diabetes.

Another finding included the presence of urinary microvesicles expressing mesangial and endothelial cell markers in the diabetic and GN groups compared to the healthy controls. Mesangial and endothelial cells are typically not exposed to the urinary space and in order to identify these microvesicles in the urine, the microvesicles would need to pass through the glomerular basement membrane (GBM) to have access to the Bowman's capsule. The usual size of slit diaphragm is about 40 nm and with an intact GBM, one would not expect to find these microvesicles in the urine.

In summary, digital flow cytometry was used to detect urinary microvesicles as a marker of podocyte injury in patients with type 2 DM and GN. These findings suggest that podocyte injury precedes proteinuria in diabetic patients, and patients with GN have more podocyte injury compared to diabetic patients with macroalbuminuria. In addition, other glomerular markers including parietal, mesangial, and endothelial cell markers were identified in the urine of patients with diabetes and GN. Urinary microvesicles can be used as biomarkers in identifying podocyte injury.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A flow cytometry method or enzyme-linked immunosorbent assay method for detecting diabetic nephropathy or glomerulonephritis, wherein said method comprises:
(a) performing flow cytometry or an enzyme-linked immunosorbent assay using a urine sample obtained from a mammal to detect the presence of an elevated level of urinary microvesicles expressing a podocyte, parietal, mesangial, or endothelial cell marker, and
(b) classifying said mammal as having diabetic nephropathy or glomerulonephritis.

2. The method of claim 1, wherein said mammal is a human.

3. The method of claim 1, wherein said method comprises detecting the presence of an elevated level of urinary microvesicles expressing a podocyte cell marker.

4. The method of claim 3, wherein said podocyte cell marker is synaptopodin, nephrin, podocin, or podocalyxin.

5. The method of claim 1, wherein said method comprises detecting the presence of an elevated level of urinary microvesicles expressing a parietal cell marker.

6. The method of claim 5, wherein said parietal cell marker is claudin-1 or CK-8.

7. The method of claim 1, wherein said method comprises detecting the presence of an elevated level of urinary microvesicles expressing a mesangial cell marker.

8. The method of claim 7, wherein said mesangial cell marker is PDGFRβ or α-SMA.

9. The method of claim 1, wherein said method comprises detecting the presence of an elevated level of urinary microvesicles expressing an endothelial cell marker.

10. The method of claim 9, wherein said endothelial cell marker is CD62E or CD31.

11. A method for detecting podocyte injury in a mammal having a diabetic nephropathy or glomerulonephritis, wherein said method comprises:
(a) detecting, in a urine sample obtained from said mammal, the presence of an elevated level of urinary microvesicles expressing a podocyte, parietal, mesangial, or endothelial cell marker, and
(b) classifying said mammal as having podocyte injury.

12. The method of claim 11, wherein said mammal is a human.

13. The method of claim 11, wherein said method comprises detecting the presence of an elevated level of urinary microvesicles expressing a podocyte cell marker.

14. The method of claim 13, wherein said podocyte cell marker is synaptopodin, nephrin, podocin, or podocalyxin.

15. The method of claim 11, wherein said method comprises detecting the presence of an elevated level of urinary microvesicles expressing a parietal cell marker.

16. The method of claim 15, wherein said parietal cell marker is claudin-1 or CK-8.

17. The method of claim 11, wherein said method comprises detecting the presence of an elevated level of urinary microvesicles expressing a mesangial cell marker.

18. The method of claim 17, wherein said mesangial cell marker is PDGFRβ or α-SMA.

19. The method of claim 11, wherein said method comprises detecting the presence of an elevated level of urinary microvesicles expressing an endothelial cell marker.

20. The method of claim 19, wherein said endothelial cell marker is CD62E or CD31.

21. The method of claim 11, wherein said method comprises performing a flow cytometry assay or an enzyme-linked immunosorbent assay to detect said presence.
